(19) United States
(12) Patent Application Publication
(10) Pub. No.: US 2025/0352529 A1
(43) Pub. Date: Nov. 20, 2025

(54) COMBINED USE OF M1 MUSCARINIC RECEPTOR AGONISTS AND M3 MUSCARINIC RECEPTOR ANTAGONISTS TO TREAT CANCER

(71) Applicants: The United States Government As Represented By The Department Of Veterans Affairs, Washington, DC (US); University of Maryland, Baltimore, Baltimore, MD (US)

(72) Inventors: Jean-Pierre Raufman, Baltimore, MD (US); Kunrong Cheng, Baltimore, MD (US); Natalia Sampaio Moura, Baltimore, MD (US); Margaret H. Sundel, Baltimore, MD (US)

(21) Appl. No.: 19/209,061

(22) Filed: May 15, 2025

Related U.S. Application Data
(60) Provisional application No. 63/647,732, filed on May 15, 2024.

(51) Int. Cl. A61K 31/4439; A61K 31/4025; A61P 35/00

(52) U.S. Cl. CPC A61K 31/4439; A61K 31/4025; A61P 35/00

(57) ABSTRACT

Disclosed are methods of administering a combination therapy, wherein the combination therapy comprises a M1 muscarinic receptor (M₁R) activator or a composition that upregulates gene expression of CHRM1; and a M3 muscarinic receptor (M₃R) inhibitor or a composition that downregulates gene expression of CHRM3. For example, disclosed are methods of treating a subject having cancer comprising administering a therapeutically effective amount of a M1 muscarinic receptor (M₁R) activator and a therapeutically effective amount of a M3 muscarinic receptor (M₃R) inhibitor to the subject having cancer.

### CROSS-REFERENCE TO RELATED APPLICATIONS

This application claims the benefit of U.S. Provisional Patent Application No. 63/647,732, filed on May 15, 2025, which is incorporated by reference herein in its entirety.

### BACKGROUND

Colorectal cancer (CRC) is the major cause of cancer death in men under age 50, accounting for ˜10% of cancers in Veterans—yearly the VA cares for >175,000 Veterans with CRC. Current therapies for advanced CRC provide transient, if any, prolongation of life—novel approaches are urgently needed.

CRC is frequently diagnosed at advanced stages when limited therapeutic options provide only transient benefit. In 2024, >50,000 Americans, many of them Veterans, died from CRC, the deadliest cancer in young men. As the tumor microenvironment plays an important role in colon neoplasia, previous work focuses on finding therapeutic targets in the enteric nervous system. Previously, cholinergic muscarinic neurotransmission was identified as such a target. Activated M3 muscarinic receptors (M₃R) induce the expression of selective matrix metalloproteinases (MMP1, 7, 10) and microRNAs (miR21, 221, 222) that promote CRC progression. In human CRC cells, downregulating expression of CHRM3, the gene encoding M₃R, or blocking M₃R activation, robustly attenuates cell proliferation, survival, and migration. In murine CRC models, deleting M₃R expression greatly diminishes colon tumor formation.

In contrast to overexpression of M₃R, M₁R expression is surprisingly diminished in CRC. Consistent with this finding, selectively activating M₁R robustly inhibits CRC cell proliferation, an action diametrically opposed to what happens when M₃R is activated. What is needed is a more effective approach.

### BRIEF SUMMARY

Disclosed herein are methods of targeting both M₁R and M₃R as an effective therapeutic approach to colorectal and other cancers.

Disclosed are methods of treating a subject having cancer comprising administering to the subject having cancer, a therapeutically effective amount of a M1 muscarinic receptor (M₁R) activator or a composition that upregulates gene expression of CHRM1 in the subject having cancer, and administering to the subject having cancer, a therapeutically effective amount of a M3 muscarinic receptor (M₃R) inhibitor or a composition that downregulates gene expression of CHRM3 in the subject having cancer.

Disclosed are methods of reducing cancer cell proliferation comprising contacting one or more cancer cells with a combination therapy, wherein the combination therapy comprises a M₁R activator or a composition that upregulates gene expression of CHRM1; and a M₃R inhibitor or a composition that downregulates gene expression of CHRM3.

Disclosed are methods of increasing efficacy of a combination therapy in a subject having cancer comprising identifying a subject having cancer as having a high CHRM1:3 ratio; and administering the combination therapy to the subject having cancer, wherein the combination therapy comprises a therapeutically effective amount of a M1 muscarinic receptor (M₁R) activator and a therapeutically effective amount of a M3 muscarinic receptor (M₃R) inhibitor, wherein administering the combination therapy to subjects having cancer identified as having a high CHRM1:3 ratio increases efficacy of the combination therapy compared to administering the combination therapy to subjects having cancer identified as having a low CHRM1:3 ratio.

Disclosed are methods of potentiating effects of M₁R activation in a subject having cancer comprising administering, to the subject, a therapeutically effective amount of a M₁R activator or a composition that upregulates gene expression of CHRM1 in the subject, and administering, to the subject, a therapeutically effective amount of a M₃R inhibitor or a composition that downregulates gene expression of CHRM13 in the subject, wherein the combination of activating M₁R and inhibiting M₃R potentiates the effects of M₁R activation.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows M₃R deficiency attenuates colon tumor number in Apc^{min/+}. Each symbol represents one 14-week old Apc^{min/+} C57BL/6 mouse; horizontal bars are means.

FIG. 2A-C show the dual deletion of M₁R and M₃R negates benefits of deleting only M₃R. FIG. 2A) Reduced tumor number in azoxymethane (AOM)-treated Chrm3^{-/-} compared to wild type (WT) mice (p<0.05). FIG. 2B) Reduced tumor volumbe in Chrm3^{-/-} compared to WT mice (P<0.005). FIG. 2C) Fewer adenocarcinomas in AOM-treated Chrm3^{-/-} compared to WT mice (P<0.001). Bars=mean±SE for 12-20 mice/group.

FIG. 3A-E show the M₁R/M₃R expression in progressive colon neoplasia. FIG. 3A) Anti-M₁R antibody stains WT but not M₁R-deficient mouse colonocytes. FIG. 3B) More intense M₃R vs. M₁R staining in ACF (arrows) from three patients. FIG. 3C) M₃R staining in cancer (dashed circle in H&E) Inset: intense M₃R staining in cells at tumor invasive front (arrows). FIG. 3D) M₁R vs. M₃R staining intensity in ACFs FIG. 3E) M₁R vs. M₃R staining intensity in colon cancers. Means±SE, n=6 FAP and 8 cancer patients. ACF, aberrant crypt foci; NL, normal cancer; CA, colon cancer.

FIG. 4 shows M₃R, but not M₁R, overexpression in CCSCs. Images show H&E (left panels) and immunofluorescent staining with DAPI (nuclear stain), CD133 (CCSC marker), M₁R and M₃R, and Merge. Dashed boxes in H&Es show areas from which images were enlarged. CD133 staining reveals scattered CCSCs (white arrow) and CCSC clusters around blood vessels (invasive font, grey arrows). Top panels: M₁R overexpression is not detected in CCSCs. Bottom panels: M₃R overexpression in CCSCs. Size bars in H&E images.

FIG. 5 shows CHRM1 vs CHRM3 expression in CRC cell lines. Chrm1 vs Chrm3 mRNA expression, measured by qPCR, was normalized to β₂-microglobulin. Data from 8 experiments were analyzed using the CT (2^{-ΔΔCT}) method. *, **p<0.05 and 0.01 vs HT-29 cells.

FIG. 6A-D show that selective activation of M₁R dose-dependently attenuates human CRC cell proliferation. FIG. 6A) Treating H508 cells for 5 days with carbachol (carb), a non-selective muscarinic receptor agonist stimulates cell proliferation measured using the WST-1 Cell Proliferation Assay (Roche). Cont, control; Carb, carbachol. Solid black circles represent values for individual replicates. FIG. 3B-C) Increasing concentrations of selective M₁R activators, McN-A-343 and xanomeline, dose-dependently inhibits H508 cell proliferation. Cells were incubated with or without agents for 5 days. N=4. FIG. 6D). Maximal proliferation responses observed with increasing concentrations of McN-A-343 in H508 (square), HT-29 (triangle), and HCT116 (circle) human CRC cells. Cells were incubated with agents for 5 days. Dose-response curves created by nonlinear regression (GraphPad Prism).

FIG. 7A-B show combining a selective M₃R inhibitor (darifenacin) with an M₁R-selective activator potentiates inhibition of cell proliferation without cell injury. FIG. 7A) Preincubating H508 CRC cells with 30 and 60 μM darifenacin shifted the xanomeline dose-response curve progressively leftward. FIG. 7B) No cell LDH leakage was detected with increasing doses of xanomeline+/−30 or 60 μM darifenacin. Triton X-100 was the positive control for LDH release. Symbols represent means+/−SEM of 3-9 replicates.

FIG. 8A-C show tumoroids derived from HT-29 cells maintain CHRM1:3 ratio. FIG. 8A) Early passage (day-7) tumoroid. FIG. 8B) Scanning electron micrograph of later passage (day-21) HT-29 cell tumoroid. FIG. 8C) HT-29 cell tumoroids express higher CHRM3 levels, measured by qPCR (GAPDH standard). Bars, mean±SEM of ≥3 experiments.

FIG. 9A-E show HT-29 human CRC cells injected into mouse sigmoid colon form invasive tumors. FIG. 9A) in situ colon showing HT-29 cell tumor and invasion (arrows). FIG. 9B) resected colon showing HT-29 cell tumor and invasion (arrows). FIG. 9C) H&E images show submucosal solid tumors. FIG. 9D) H&E images show tumor emboli in lymphatics. FIG. 9E) H&E images show lymph node infiltration.

FIG. 10 shows that the muscarinic receptor subtype mRNA expression in intestinal mucosa from Chrm1 and Chrm3 CKO vs control mice. Relative expression of Chrm1, 2, 3, 4, and 5 mRNA in extracts of intestinal mucosal scrapings from M₁R and M₃R CKO and littermate control mice. mRNA measured by qPCR was normalized to Gapdh mRNA expression using Chrm1 mRNA in control mouse brain as a comparator. N=5 F/6 M, M₁R and 6 F/6 M M₃R CKO and the same n for M/F control mice. Bars, mean±SEM. Negligible Chrm1 and Chrm3 expression in respective CKO is from non-villin-containing cells (e.g., immunocytes).

FIG. 11 shows xanomeline impacts proliferation of CRC cell lines. Comparison of maximal inhibition of cell proliferation by increasing concentrations of xanomeline in H508 and HCT116 colon cancer cells. Compared to H508 cells, HCT116 cell proliferation was attenuated at lower xanomeline concentrations. Symbols and error bars represent means+/−SEM of 3-6 replicates.

FIG. 12A-C show the effect of combining Trospium or Darifenacin with M₁R-selective agonist on H508 cells. FIG. 12A) Nonselective MR inhibition with 5-50 μM trospium did not alter cell proliferation. FIG. 12B) Preincubating cells with 50 μM trospium did not shift the xanomeline (5-50 μM) dose-response curve. FIG. 12C) Treating cells with 10-200 μM darifenacin, a selective M₃R antagonist, progressively inhibited cell proliferation (IC50 84.0 μM). (p<0.0005). Symbols and error bars represent means+/−SEM of 3-9 replicates.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a M₁R activator” includes a plurality of such activators, reference to “the M₃R inhibitor” is a reference to one or more inhibitors and equivalents thereof known to those skilled in the art, and so forth.

The word “or” as used herein means any one member of a particular list and also includes any combination of members of that list.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition (e.g. colorectal cancer, pancreatic cancer, or gastric cancer). For example, “treating” colorectal cancer may refer to inhibiting survival, growth, and/or spread of the cancer. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, a “M₁R activator” or “M₁R agonist” refers to any agent, (e.g., a small molecule) capable of activating M₁R. M₁R activators can include selective M₁R activators and activators that also inhibit other proteins, such as other muscarinic receptor subtypes (e.g. M₂R, M₃R, M₄R, or M₅R). In some embodiments, M₁R activators will selectively activate M₁R, with a selectivity ratio greater of at least about 10-fold, such as greater than at least about 30-fold, for inhibition of M₁R relative to another muscarinic receptor subtype. In some aspects, a selective M₁R activator specifically increases the activity of M₁R with minimal or no effect on other muscarinic receptor subtypes. Exemplary M₁R activators include, but are not limited to, nucleic acids, DNA, RNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the M₁R activator is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. In some aspects, M₁R activators include natural products, derivatives, and analogs thereof. Accordingly, compounds said to be “capable of activating” a particular protein (e.g., M₁R) comprise any such activator.

As used herein, a “M₃R inhibitor” or “M₃R antagonist” refers to any agent, (e.g., a small molecule) capable of inhibiting M₃R or blocking activation of M₃R. M₃R inhibitors can include selective M₃R inhibitors and inhibitors that also inhibit other proteins, such as other muscarinic receptor subtypes (e.g. M₁R, M₂R, M₄R, or M₅R). In some embodiments, M₃R inhibitors will selectively inhibit M₃R, with a selectivity ratio greater of at least about 10-fold, such as greater than at least about 30-fold, for inhibition of M₃R relative to another muscarinic receptor subtype. In some aspects, a selective M₃R inhibitor specifically blocks the activity of M₃R with minimal or no effect on other muscarinic receptor subtypes. In some aspects, a M₃R inhibitor can inhibit by competitive, uncompetitive, or non-competitive means. With respect to its binding mechanism, a M₃R inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the M₃R inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. In some aspects, M₃R inhibitors include natural products, derivatives, and analogs thereof. In some embodiments, the M₃R inhibitor can be nucleic acid molecules including, but not limited to, siRNA that reduce the amount of functional protein in a cell. Accordingly, compounds said to be “capable of inhibiting” a particular protein (e.g., M₃R) comprise any such inhibitor.

As used herein, “cholinergic receptor muscarinic 1” or “CHRM1” is the gene that encodes muscarinic acetylcholine receptor M1 (M₁R). CHRM1 is localized to 11q13. The nucleic acid sequence of CHRM1 can be found at accession no NM_000738. The amino acid sequence of CHRM1 can be found as reference sequence NP_000729.2.

As used herein, “cholinergic receptor muscarinic 3” or “CHRM3” is the gene that encodes muscarinic acetylcholine receptor M3 (M₃R). The nucleic acid sequence of CHRM3 can be found at accession no. NM_000740. The amino acid sequence of CHRM3 can be found as reference sequence NP_000731.1.

As used herein, the term “combination therapy” refers to a method (e.g. of treatment) comprising administering to a subject at least two therapeutic agents, optionally as one or more pharmaceutical compositions. For example, “combination therapy” refers to a method (e.g. of treatment) comprising administering to a subject at least two therapeutic agents, optionally as one or more pharmaceutical compositions, wherein the at least two therapeutic agents comprise one therapeutic agent that is a M₁R activator or a composition that upregulates gene expression of CHRM1 and one therapeutic agent that is a M₃R inhibitor or a composition that downregulates gene expression of CHRM3. For example, a combination therapy may comprise administration of a single pharmaceutical composition comprising at least two therapeutic agents and one or more pharmaceutically acceptable carrier, excipient, diluent, and/or surfactant. A combination therapy may comprise administration of two or more pharmaceutical compositions, each composition comprising one or more therapeutic agent and one or more pharmaceutically acceptable carrier, excipient, diluent, and/or surfactant. In various embodiments, at least one of the therapeutic agents is a M₁R activator. In various embodiments, at least one of the therapeutic agents is a M₃R inhibitor. The two agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due an additive or synergistic effect of combining the two or more therapeutics.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

The muscarinic cholinergic receptors belong to a larger family of G protein-coupled receptors. The functional diversity of these receptors is defined by the binding of acetylcholine and includes cellular responses such as adenylate cyclase inhibition, phosphoinositide degeneration, and potassium channel mediation. Muscarinic receptors influence many effects of acetylcholine in the central and peripheral nervous system. The muscarinic cholinergic receptor 1 (M₁R) is involved in mediation of vagally-induced bronchoconstriction and in the acid secretion of the gastrointestinal tract. The gene encoding this receptor is localized to 11q13. The muscarinic cholinergic receptor 3 controls smooth muscle contraction and its stimulation causes secretion of glandular tissue.

Disclosed are methods of administering a combination therapy to a subject in need thereof, wherein the combination therapy comprises a M1 muscarinic receptor (M₁R) activator or a composition that upregulates gene expression of CHRM1; and a M3 muscarinic receptor (M₃R) inhibitor or a composition that downregulates gene expression of CHRM3.

Disclosed are methods of administering a combination therapy to a subject in need thereof, wherein the combination therapy comprises a M1 muscarinic receptor (M₁R) activator and a M3 muscarinic receptor (M₃R) inhibitor.

Disclosed are methods of administering a combination therapy to a subject in need thereof, wherein the combination therapy comprises a composition that upregulates gene expression of CHRM1; and a composition that downregulates gene expression of CHRM3.

Disclosed are methods of administering to a subject having cancer, a M1 muscarinic receptor (MIR) activator or a composition that upregulates gene expression of CHRM1 and administering a M3 muscarinic receptor (M₃R) inhibitor or a composition that downregulates gene expression of CHRM3.

Disclosed are methods of administering to a subject having cancer, a M1 muscarinic receptor (MIR) activator and a M3 muscarinic receptor (M₃R) inhibitor.

Disclosed are methods of administering to a subject having cancer, a composition that upregulates gene expression of CHRM1 in the subject having cancer and a composition that downregulates gene expression of CHRM3.

In some aspects, the disclosed methods can comprise administering a M₁R activator, a composition that upregulates gene expression of CHRM1, a M₃R inhibitor, and a composition that downregulates gene expression of CHRM3 to a subject in need thereof (e.g. a subject having cancer). Thus, in some aspects, the methods involve altering expression of CHRM1 or CHRM3 at the genetic level and activity of M₁R or M₃R at the receptor level. In some aspects, the methods involve altering a combination of expression of CHRM1 or CHRM3 at the genetic level and/or activity of M₁R or M₃R at the receptor level.

### 1. Methods of Treating

Disclosed are methods of treating a subject having cancer comprising administering, to the subject having cancer, a therapeutically effective amount of a M1 muscarinic receptor (M₁R) activator or a composition that upregulates gene expression of CHRM1 in the subject having cancer, and administering, to the subject having cancer, a therapeutically effective amount of a M3 muscarinic receptor (M₃R) inhibitor or a composition that downregulates gene expression of CHRM3 in the subject having cancer.

Disclosed are methods of treating a subject having cancer comprising administering a therapeutically effective amount of a M1 muscarinic receptor (M₁R) activator and a therapeutically effective amount of a M3 muscarinic receptor (M₃R) inhibitor to the subject having cancer.

Disclosed are methods of treating a subject having cancer comprising administering a composition that upregulates gene expression of CHRM1 in the subject having cancer and a composition that downregulates gene expression of CHRM3 in the subject having cancer.

In some aspects, the M₁R activator is a selective M₁R activator. Thus, in some aspects, the selective M₁R activator specifically targets M₁R with little to no effects on any other subtype of muscarinic receptor. In some aspects, the selective M₁R activator can be, but is not limited to, Xanomeline (Orthosteric M1/M4-preferring agonist), VU0357017 (high selectivity), 1-Trifluoromethylbenzyl-4-(2-hydroxyethyl)piperazine (TBPB) (moderate-high selectivity), 77-LH-28-1 (moderate selectivity) or McN-A-343 (high selectivity). In some aspects, a M₁R activator can be, but is not limited to, Talsaclidine, HTL-9936, AF102B, PIPE-307, 77-LH-28-1, GSK-5, Acetylcholine, Arecoline, Carbachol, Cevimeline, Itameline, Muscarine, Oxotremorine, Pilocarpine, Vedaclidine, CDD-0097, L-689, L-660, BQCA, BQZ-12, VU-0090157, VU-0029767, VU0467319, or [3H]PT-1284.

In some aspects, the M₃R inhibitor is a selective M₃R inhibitor. Thus, in some aspects, the selective M₃R inhibitor specifically targets M₃R with little to no effects on any other subtype of muscarinic receptor. In some aspects, the selective M₃R inhibitor can be, but is not limited to, Darifenacin (high selectivity), Solifenacin (moderate-high selectivity), Tiotropium (functional selectivity), and Glycopyrronium (Glycopyrrolate) (moderate selectivity). In some aspects, a M₃R activator can be, but is not limited to, atropine, AZD9164, tramadol, hyoscyamine, aclidinium bromide, 4-DAMP (1,1-Dimethyl-4-diphenylacetoxypiperidinium iodide), diphenhydramine, fluoxetine, DAU-5884 (8-Methyl-8-azabicyclo-3-endo[1.2.3]oct-3-yl-1,4-dihydro-2-oxo-3(2H)-quinazolinecarboxylic acid ester), HL-031,120 ((3R,2′R)-enantiomer of EA-3167), ipratropium, J-104,129 ((aR)-a-Cyclopentyl-a-hydroxy-N-[1-(4-methyl-3-pentenyl)-4-piperidinyl]benzeneacetamide), oxybutynin, procyclidine, tiotropium, tolterodine, zamifenacin ((3R)-1-[2-(1-,3-Benzodioxol-5-yl)ethyl]-3-(diphenylmethoxy)piperidine), solifenacin, Imidafenacin, Oxybutynin, Tiotropium, or Ipratropium.

In some aspects, the subject having cancer or a subject in need of the treatments disclosed herein has a high CHRM1:3 ratio (e.g. 0.7 or greater).

In some aspects, the CHRM1:3 ratio of the subject having cancer can be used to determine if a treatment, such as the disclosed combination therapies, will be effective. In some aspects, the CHRM1:3 ratio of the subject having cancer can be used to determine if the subject will respond to a treatment, such as the disclosed combination therapies. Thus, in some aspects, a subject's CHRM1:3 ratio can have predictive value. In some aspects, a subject having higher levels of CHRM1 mRNA compared to CHRM3 mRNA results in higher efficacy of a selective M₁R activator when treated in combination with a M₃R inhibitor. In some aspects, subjects can be characterized as having a high CHRM1:3 ratio, intermediate CHRM1:3 ratio, or low CHRM1:3 ratio. In some aspects, a high CHRM1:3 ratio is 0.7 or greater. In some aspects, an intermediate CHRM1:3 ratio. In some aspects, a low CHRM1:3 ratio is 0.0 (or less) to 0.3. In some aspects, a high CHRM1:3 ratio means the subject has at least a 1 fold or at least a 1.5 fold increase of CHRM1 compared to CHRM3. In some aspects, an intermediate CHRM1:3 ratio means the subject has about a zero to 1 fold increase or about a zero to 1.5 fold increase of CHRM1 compared to CHRM3. In some aspects, a low CHRM1:3 ratio means the subject has at least a 0.5 fold increase of CHRM3 compared to CHRM1.

In some aspects, the subject having cancer has a high CHRM1:3 ratio. In some aspects, the disclosed methods of treating can further comprise a step of identifying a subject having cancer as having a high CHRM1:3 ratio. In some aspects, the presence of a high CHRM1:3 ratio is predictive that the subject will respond to and/or have better efficacy with the disclosed combination therapies.

In some aspects, the subject has cancer. In some aspects, the cancer is colorectal cancer. Thus, in the disclosed methods, in some aspects the subject having cancer is a subject having colorectal cancer. In some aspects, the cancer is colon cancer. In some aspects, the cancer is any cancer associated with muscarinic receptors, for example, but not limited to, lung cancer, prostate cancer, breast cancer, gastric cancer, pancreatic cancer, or bladder cancer.

In some aspects, treating cancer results in a decrease in proliferation of cancer cells in the subject. In some aspects of the disclosed methods, cell toxicity does not occur. Thus, in some aspects, the effects of the combination therapy on cell proliferation are mediated by post-muscarinic receptor signal transduction and not by cell toxicity.

In some aspects, the disclosed methods can comprise genetically altering CHRM1 or CHRM3. For example, in some aspects, gene expression of CHRM1 or CHRM3 can be altered at the DNA or RNA level.

In some aspects, a composition that downregulates gene expression of CHRM3 can be a composition that knocks out all or a portion of CHRM3. In some aspects, a composition that knocks out all or a portion of CHRM3 comprises a CRISPR-Cas9 system that targets CHRM3. For example, a target site in CHRM3 or just outside of the CHRM3 coding sequence can be targeted with a gRNA which allows for Cas9 to cleave in or around CHRM3, thus removing all or a portion of CHRM3. In some aspects, if a portion of CHRM3 is removed, the portion removed results in a nonfunctional M₃R. In some aspects, known CRISPR-Cas9 techniques can be used to determine a gRNA that binds to a target site in CHRM3 or just outside of the CHRM3 coding sequence.

In some aspects, a composition that downregulates gene expression of CHRM3 can be a composition comprising a transcription activator-like effector nuclease (TALEN). In some aspects, the TALEN can introduce double-strand breaks in the DNA around or within CHRM3, triggering error prone DNA repair that disrupts gene function.

In some aspects, a composition that downregulates gene expression of CHRM3 can be, but is not limited to, a short hairpin RNA (shRNA), or RNA interference (RNAi) that targets CHRM3 mRNA. In some aspects, RNAi uses a small interfering RNA (siRNA) to silence gene expression. Thus, in some aspects, downregulating gene expression of CHRM3 can involve degrading CHRM3 mRNA.

In some aspects, a composition that upregulates gene expression CHRM1 can be a transcription activator that activates a CHRM1 promoter. In some aspects, increasing gene expression of CHRM1 can lead to increased M₁R which can allow for a more efficient treatment with the disclosed combination therapy.

### 2. Methods of Reducing Cancer Cell Proliferation

Disclosed are methods of reducing cancer cell proliferation comprising contacting one or more cancer cells with a combination therapy, wherein the combination therapy comprises a M₁R activator or a composition that upregulates gene expression of CHRM1; and a M₃R inhibitor or a composition that downregulates gene expression of CHRM3.

Disclosed are methods of reducing cancer cell proliferation comprising contacting one or more cancer cells with a combination of a M1 muscarinic receptor (M₁R) activator and a M3 muscarinic receptor (M₃R) inhibitor.

Disclosed are methods of reducing cancer cell proliferation comprising contacting one or more cancer cells with a composition that upregulates gene expression of CHRM1 in the subject having cancer and a composition that downregulates gene expression of CHRM3 in the subject having cancer.

In some aspects, the disclosed methods of reducing cancer cell proliferation can be performed in vitro or in vivo. Thus, in some aspects, the cancer cells are in a subject. In some aspects, the cancer cells are in a cell culture container. In some aspects, when the cancer cells are in a subject, the step of contacting one or more cancer cells comprises administering, to the subject, a therapeutically effective amount of a M1 muscarinic receptor (M₁R) activator or a composition that upregulates gene expression of CHRM1 in the subject, and administering, to the subject, a therapeutically effective amount of a M3 muscarinic receptor (M₃R) inhibitor or a composition that downregulates gene expression of CHRM3 in the subject.

In some aspects, the M₁R activator is a selective M₁R activator. Thus, in some aspects, the selective M₁R activator specifically targets M₁R with little to no effects on any other subtype of muscarinic receptor. In some aspects, the selective M₁R activator can be, but is not limited to, Xanomeline (Orthosteric M1/M4-preferring agonist), VU0357017 (high selectivity), 1-Trifluoromethylbenzyl-4-(2-hydroxyethyl)piperazine (TBPB) (moderate-high selectivity), 77-LH-28-1 (moderate selectivity), or McN-A-343 (high selectivity). In some aspects, a M₁R activator can be, but is not limited to, Talsaclidine, HTL-9936, AF102B, PIPE-307, 77-LH-28-1, GSK-5, Acetylcholine, Arecoline, Carbachol, Cevimeline, Itameline, Muscarine, Oxotremorine, Pilocarpine, Vedaclidine, CDD-0097, L-689, L-660, BQCA, BQZ-12, VU-0090157, VU-0029767, VU0467319, or [3H]PT-1284.

In some aspects, the M₃R inhibitor is a selective M₃R inhibitor. Thus, in some aspects, the selective M₃R inhibitor specifically targets M₃R with little to no effects on any other subtype of muscarinic receptor. In some aspects, the selective M₃R inhibitor can be, but is not limited to, Darifenacin (high selectivity), Solifenacin (moderate-high selectivity), Tiotropium (functional selectivity), and Glycopyrronium (Glycopyrrolate) (moderate selectivity). In some aspects, a M₃R activator can be, but is not limited to, atropine, AZD9164, tramadol, hyoscyamine, aclidinium bromide, 4-DAMP (1,1-Dimethyl-4-diphenylacetoxypiperidinium iodide), diphenhydramine, fluoxetine, DAU-5884 (8-Methyl-8-azabicyclo-3-endo[1.2.3]oct-3-yl-1,4-dihydro-2-oxo-3(2H)-quinazolinecarboxylic acid ester), HL-031,120 ((3R,2′R)-enantiomer of EA-3167), ipratropium, J-104,129 ((aR)-a-Cyclopentyl-a-hydroxy-N-[1-(4-methyl-3-pentenyl)-4-piperidinyl]benzeneacetamide), oxybutynin, procyclidine, tiotropium, tolterodine, zamifenacin ((3R)-1-[2-(1-,3-Benzodioxol-5-yl)ethyl]-3-(diphenylmethoxy)piperidine), solifenacin, Imidafenacin, Oxybutynin, Tiotropium, or Ipratropium.

In some aspects, the subject having cancer or a subject in need of the treatments disclosed herein has a high CHRM1:3 ratio (e.g. 0.7 or greater).

In some aspects, the CHRM1:3 ratio of the subject having cancer can be used to determine if a treatment, such as the disclosed combination therapies, will be effective. In some aspects, the CHRM1:3 ratio of the subject having cancer can be used to determine if the subject will respond to a treatment, such as the disclosed combination therapies. Thus, in some aspects, a subject's CHRM1:3 ratio can have predictive value. In some aspects, a subject having higher levels of CHRM1 mRNA compared to CHRM3 mRNA results in higher efficacy of a selective M₁R activator when treated in combination with a M₃R inhibitor.

In some aspects, subjects can be characterized as having a high CHRM1:3 ratio, intermediate CHRM1:3 ratio, or low CHRM1:3 ratio. In some aspects, a high CHRM1:3 ratio is 0.7 or greater. In some aspects, an intermediate CHRM1:3 ratio. In some aspects, a low CHRM1:3 ratio is 0.0 (or less) to 0.3. In some aspects, a high CHRM1:3 ratio means the subject has at least a 1 fold or at least a 1.5 fold increase of CHRM1 compared to CHRM3. In some aspects, an intermediate CHRM1:3 ratio means the subject has about a zero to 1 fold increase or about a zero to 1.5 fold increase of CHRM1 compared to CHRM3. In some aspects, a low CHRM1:3 ratio means the subject has at least a 0.5 fold increase of CHRM3 compared to CHRM1.

In some aspects, the subject having cancer has a high CHRM1:3 ratio. In some aspects, the disclosed methods of reducing cancer cell proliferation can further comprise a step of identifying a subject having cancer as having a high CHRM1:3 ratio. In some aspects, the presence of a high CHRM1:3 ratio is predictive that the subject will respond and/or have better efficacy (e.g. reduction in cancer cell proliferation) with the disclosed combination therapies.

In some aspects, the cancer cells are colorectal cancer cells. In some aspects, the cancer cells are colon cancer cells. In some aspects, the cancer cells are derived from any cancer associated with muscarinic receptors, for example, but not limited to, lung cancer, prostate cancer, breast cancer, gastric cancer, pancreatic cancer, or bladder cancer.

In some aspects, the reduced cancer cell proliferation does not result in cell toxicity. Thus, in some aspects, the effects of the combination therapy on cell proliferation are mediated by post-muscarinic receptor signal transduction and not by cell toxicity.

In some aspects, the disclosed methods can comprise genetically altering CHRM1 or CHRM3. For example, in some aspects, gene expression of CHRM1 or CHRM3 can be altered at the DNA or RNA level.

In some aspects, a composition that downregulates gene expression of CHRM3 can be a composition that knocks out all or a portion of CHRM3. In some aspects, a composition that knocks out all or a portion of CHRM3 comprises a CRISPR-Cas9 system that targets CHRM3. For example, a target site in CHRM3 or just outside of the CHRM3 coding sequence can be targeted with a gRNA which allows for Cas9 to cleave in or around CHRM3, thus removing all or a portion of CHRM3. In some aspects, if a portion of CHRM3 is removed, the portion removed results in a nonfunctional M₃R. In some aspects, known CRISPR-Cas9 techniques can be used to determine a gRNA that binds to a target site in CHRM3 or just outside of the CHRM3 coding sequence.

In some aspects, a composition that downregulates gene expression of CHRM3 can be a composition comprising a transcription activator-like effector nuclease (TALEN). In some aspects, the TALEN can introduce double-strand breaks in the DNA around or within CHRM3, triggering error prone DNA repair that disrupts gene function.

In some aspects, a composition that downregulates gene expression of CHRM3 can be, but is not limited to, a short hairpin RNA (shRNA), or RNA interference (RNAi) that targets CHRM3 mRNA. In some aspects, RNAi uses a small interfering RNA (siRNA) to silence gene expression. Thus, in some aspects, downregulating gene expression of CHRM3 can involve degrading CHRM3 mRNA.

In some aspects, a composition that upregulates gene expression CHRM1 can be a transcription activator that activates a CHRM1 promoter. In some aspects, increasing gene expression of CHRM1 can lead to increased M₁R which can allow for a more efficient treatment with the disclosed combination therapy.

### 3. Methods of Increasing Efficacy of Combination Therapy

In some aspects, the CHRM1:3 ratio of a subject having cancer can be used to determine if a treatment, such as the disclosed combination therapies, will be effective. In some aspects, a subject's CHRM1:3 ratio can have predictive value.

Disclosed are methods of increasing efficacy of a combination therapy in a subject having cancer comprising identifying a subject having cancer as having a high CHRM1:3 ratio; and administering the combination therapy to the subject having cancer, wherein the combination therapy comprises a therapeutically effective amount of a M1 muscarinic receptor (M₁R) activator and a therapeutically effective amount of a M3 muscarinic receptor (M₃R) inhibitor, wherein administering the combination therapy to subjects having cancer identified as having a high CHRM1:3 ratio increases efficacy of the combination therapy compared to administering the combination therapy to subjects having cancer identified as having a low CHRM1:3 ratio.

In some aspects, the subject having cancer or a subject in need of the treatments disclosed herein has a high CHRM1:3 ratio (e.g. 0.7 or greater).

In some aspects, identifying a subject having cancer as having a high CHRM1:3 ratio comprises measuring the mRNA levels of each of CHRM1 and CHRM3 in the subject. In some aspects, identifying a subject having cancer as having a high CHRM1:3 ratio comprises measuring the DNA levels of each of CHRM1 and CHRM3.

In some aspects, the M₁R activator is a selective M₁R activator. Thus, in some aspects, the selective M₁R activator specifically targets M₁R with little to no effects on any other subtype of muscarinic receptor. In some aspects, the selective M₁R activator can be, but is not limited to, Xanomeline (Orthosteric M1/M4-preferring agonist), VU0357017 (high selectivity), 1-Trifluoromethylbenzyl-4-(2-hydroxyethyl)piperazine (TBPB) (moderate-high selectivity), 77-LH-28-1 (moderate selectivity), or McN-A-343 (high selectivity). In some aspects, a M₁R activator can be, but is not limited to, Talsaclidine, HTL-9936, AF102B, PIPE-307, 77-LH-28-1, GSK-5, Acetylcholine, Arecoline, Carbachol, Cevimeline, Itameline, Muscarine, Oxotremorine, Pilocarpine, Vedaclidine, CDD-0097, L-689, L-660, BQCA, BQZ-12, VU-0090157, VU-0029767, VU0467319, or [3H]PT-1284. In some aspects, the M₃R inhibitor is a selective M₃R inhibitor. Thus, in some aspects, the selective M₃R inhibitor specifically targets M₃R with little to no effects on any other subtype of muscarinic receptor. In some aspects, the selective M₃R inhibitor can be, but is not limited to, Darifenacin (high selectivity), Solifenacin (moderate-high selectivity), Tiotropium (functional selectivity), and Glycopyrronium (Glycopyrrolate) (moderate selectivity). In some aspects, a M₃R activator can be, but is not limited to, atropine, AZD9164, tramadol, hyoscyamine, aclidinium bromide, 4-DAMP (1,1-Dimethyl-4-diphenylacetoxypiperidinium iodide), diphenhydramine, fluoxetine, DAU-5884 (8-Methyl-8-azabicyclo-3-endo[1.2.3]oct-3-yl-1,4-dihydro-2-oxo-3(2H)-quinazolinecarboxylic acid ester), HL-031,120 ((3R,2′R)-enantiomer of EA-3167), ipratropium, J-104,129 ((aR)-a-Cyclopentyl-a-hydroxy-N-[1-(4-methyl-3-pentenyl)-4-piperidinyl]benzeneacetamide), oxybutynin, procyclidine, tiotropium, tolterodine, zamifenacin ((3R)-1-[2-(1-,3-Benzodioxol-5-yl)ethyl]-3-(diphenylmethoxy)piperidine), solifenacin, Imidafenacin, Oxybutynin, Tiotropium, or Ipratropium.

In some aspects, the cancer is colorectal cancer. Thus, in the disclosed methods, in some aspects the subject having cancer is a subject having colorectal cancer. In some aspects, the cancer is colon cancer. In some aspects, the cancer is any cancer associated with muscarinic receptors, for example, but not limited to, lung cancer, prostate cancer, breast cancer, gastric cancer, pancreatic cancer, or bladder cancer.

In some aspects, increased efficacy of the combination therapy results in a decrease in proliferation of cancer cells in the subject. In some aspects, cell toxicity does not occur. Thus, in some aspects, the effects of the combination therapy on cell proliferation are mediated by post-muscarinic receptor signal transduction and not by cell toxicity.

### 4. Methods of Potentiating Effects of M₁R Activation

Disclosed are methods of potentiating effects of M₁R activation in a subject having cancer comprising administering, to the subject, a therapeutically effective amount of a M₁R activator or a composition that upregulates gene expression of CHRM1 in the subject, and administering, to the subject, a therapeutically effective amount of a M₃R inhibitor or a composition that downregulates gene expression of CHRM3 in the subject, wherein the combination of activating M₁R and inhibiting M₃R potentiates the effects of M₁R activation.

In some aspects, the effects of M₁R activation are reducing cell proliferation, thus, in some aspects, the combination of activating M₁R and inhibiting M₃R potentiates the reduction of cell proliferation.

In some aspects, the M₁R activator is a selective M₁R activator. Thus, in some aspects, the selective M₁R activator specifically targets M₁R with little to no effects on any other subtype of muscarinic receptor. In some aspects, the selective M₁R activator can be, but is not limited to, Xanomeline (Orthosteric M1/M4-preferring agonist), VU0357017 (high selectivity), 1-Trifluoromethylbenzyl-4-(2-hydroxyethyl)piperazine (TBPB) (moderate-high selectivity), 77-LH-28-1 (moderate selectivity), or McN-A-343 (high selectivity). In some aspects, a M₁R activator can be, but is not limited to, Talsaclidine, HTL-9936, AF102B, PIPE-307, 77-LH-28-1, GSK-5, Acetylcholine, Arecoline, Carbachol, Cevimeline, Itameline, Muscarine, Oxotremorine, Pilocarpine, Vedaclidine, CDD-0097, L-689, L-660, BQCA, BQZ-12, VU-0090157, VU-0029767, VU0467319, or [3H]PT-1284.

In some aspects, the M₃R inhibitor is a selective M₃R inhibitor. Thus, in some aspects, the selective M₃R inhibitor specifically targets M₃R with little to no effects on any other subtype of muscarinic receptor. In some aspects, the selective M₃R inhibitor can be, but is not limited to, Darifenacin (high selectivity), Solifenacin (moderate-high selectivity), Tiotropium (functional selectivity), and Glycopyrronium (Glycopyrrolate) (moderate selectivity). In some aspects, a M₃R activator can be, but is not limited to, atropine, AZD9164, tramadol, hyoscyamine, aclidinium bromide, 4-DAMP (1,1-Dimethyl-4-diphenylacetoxypiperidinium iodide), diphenhydramine, fluoxetine, DAU-5884 (8-Methyl-8-azabicyclo-3-endo[1.2.3]oct-3-yl-1,4-dihydro-2-oxo-3(2H)-quinazolinecarboxylic acid ester), HL-031,120 ((3R,2′R)-enantiomer of EA-3167), ipratropium, J-104,129 ((aR)-a-Cyclopentyl-a-hydroxy-N-[1-(4-methyl-3-pentenyl)-4-piperidinyl]benzeneacetamide), oxybutynin, procyclidine, tiotropium, tolterodine, zamifenacin ((3R)-1-[2-(1-,3-Benzodioxol-5-yl)ethyl]-3-(diphenylmethoxy)piperidine), solifenacin, Imidafenacin, Oxybutynin, Tiotropium, or Ipratropium.

In some aspects, the subject having cancer or a subject in need of the treatments disclosed herein has a high CHRM1:3 ratio (e.g. 0.7 or greater).

In some aspects, the CHRM1:3 ratio of the subject having cancer can be used to determine if a treatment, such as the disclosed combination therapies, will be effective. In some aspects, the CHRM1:3 ratio of the subject having cancer can be used to determine if the subject will respond to a treatment, such as the disclosed combination therapies. Thus, in some aspects, a subject's CHRM1:3 ratio can have predictive value. In some aspects, a subject having higher levels of CHRM1 mRNA compared to CHRM3 mRNA results in higher efficacy of a selective M₁R activator when treated in combination with a M₃R inhibitor. In some aspects, subjects can be characterized as having a high CHRM1:3 ratio, intermediate CHRM1:3 ratio, or low CHRM1:3 ratio. In some aspects, a high CHRM1:3 ratio is 0.7 or greater. In some aspects, an intermediate CHRM1:3 ratio. In some aspects, a low CHRM1:3 ratio is 0.0 (or less) to 0.3. In some aspects, a high CHRM1:3 ratio means the subject has at least a 1 fold or at least a 1.5 fold increase of CHRM1 compared to CHRM3. In some aspects, an intermediate CHRM1:3 ratio means the subject has about a zero to 1 fold increase or about a zero to 1.5 fold increase of CHRM1 compared to CHRM3. In some aspects, a low CHRM1:3 ratio means the subject has at least a 0.5 fold increase of CHRM3 compared to CHRM1.

In some aspects, the subject having cancer has a high CHRM1:3 ratio. In some aspects, the disclosed methods can further comprise a step of identifying a subject having cancer as having a high CHRM1:3 ratio. In some aspects, the presence of a high CHRM1:3 ratio is predictive that the subject will respond and/or have better efficacy with the disclosed combination therapies.

In some aspects, the cancer is colorectal cancer. Thus, in the disclosed methods, in some aspects the subject having cancer is a subject having colorectal cancer. In some aspects, the cancer is colon cancer. In some aspects, the cancer is any cancer associated with muscarinic receptors, for example, but not limited to, lung cancer, prostate cancer, breast cancer, gastric cancer, pancreatic cancer, or bladder cancer.

In some aspects, potentiating effects of M₁R activation results in a greater decrease in proliferation of cancer cells in the subject having cancer compared to a subject having cancer administered only a therapeutically effective amount of a M₁R activator. Thus, in some aspects, administering only a therapeutically effective amount of a M₁R activator can reduce cancer cell proliferation, however, the combination of a therapeutically effective amount of a M₁R activator and a therapeutically effective amount of a M₃R inhibitor potentiates the reduction in cancer cell proliferation.

In some aspects, the disclosed methods can comprise genetically altering CHRM1 or CHRM3. For example, in some aspects, gene expression of CHRM1 or CHRM3 can be altered at the DNA or RNA level.

In some aspects, a composition that downregulates gene expression of CHRM3 can be a composition that knocks out all or a portion of CHRM3. In some aspects, a composition that knocks out all or a portion of CHRM3 comprises a CRISPR-Cas9 system that targets CHRM3. For example, a target site in CHRM3 or just outside of the CHRM3 coding sequence can be targeted with a gRNA which allows for Cas9 to cleave in or around CHRM3, thus removing all or a portion of CHRM3. In some aspects, if a portion of CHRM3 is removed, the portion removed results in a nonfunctional M₃R. In some aspects, known CRISPR-Cas9 techniques can be used to determine a gRNA that binds to a target site in CHRM3 or just outside of the CHRM3 coding sequence.

In some aspects, a composition that downregulates gene expression of CHRM3 can be a composition comprising a transcription activator-like effector nuclease (TALEN). In some aspects, the TALEN can introduce double-strand breaks in the DNA around or within CHRM3, triggering error prone DNA repair that disrupts gene function.

In some aspects, a composition that downregulates gene expression of CHRM3 can be, but is not limited to, a short hairpin RNA (shRNA), or RNA interference (RNAi) that targets CHRM3 mRNA. In some aspects, RNAi uses a small interfering RNA (siRNA) to silence gene expression. Thus, in some aspects, downregulating gene expression of CHRM3 can involve degrading CHRM3 mRNA.

In some aspects, a composition that upregulates gene expression CHRM1 can be a transcription activator that activates a CHRM1 promoter. In some aspects, increasing gene expression of CHRM1 can lead to increased M₁R which can allow for a more efficient treatment with the disclosed combination therapy.

### C. Compositions

Disclosed are compositions comprising any of the disclosed therapeutics. Disclosed are compositions comprising a M₁R activator or a composition that upregulates gene expression of CHRM1. Disclosed are compositions comprising a M₃R inhibitor or a composition that downregulates gene expression of CHRM3. In some aspects, disclosed are compositions comprising both a M₁R activator and a M₃R inhibitor. In some aspects, disclosed are compositions comprising both a composition that upregulates gene expression of CHRM1 and a composition that downregulates gene expression of CHRM3.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The disclosed activators and inhibitors can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed activators or inhibitors described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the peptides disclosed herein, and a pharmaceutically acceptable carrier. In one aspect, disclosed are pharmaceutical compositions comprising the disclosed peptides. That is, a pharmaceutical composition can be provided comprising a therapeutically effective amount of at least one disclosed peptide or at least one product of a disclosed method and a pharmaceutically acceptable carrier.

In certain aspects, the disclosed pharmaceutical compositions comprise the disclosed M₃R inhibitors (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the activators and inhibitors described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention can comprise a disclosed M₃R inhibitor (or pharmaceutically acceptable salts thereof) or a disclosed M₁R activator (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### D. Combination Therapy Plus Known Cancer Therapeutic

In one aspect of the disclosed methods, the combination therapy can be administered alone or in combination with one or more additional therapeutic agents. The additional therapeutic agents are selected based on the disease or symptom to be treated. Thus, in some aspects, the additional therapeutic agent can be any known cancer agent, specifically a known therapeutic agent for the cancers described herein. A description of the various classes of suitable pharmacological agents and drugs may be found in Goodman and Gilman, The Pharmacological Basis of Therapeutics, (11th Ed., McGraw-Hill Publishing Co.) (2005).

In some aspects, the additional therapeutic agent can be an anti-inflammatory.

### E. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising a M₁R activator and a M₃R inhibitor, or a composition that upregulates gene expression of CHRM1 and a composition that downregulates gene expression of CHRM3. In some aspects, the disclosed kits can comprise any of the disclosed compositions.

### F. Embodiments

Embodiment 1: A method of treating a subject having cancer comprising administering, to the subject having cancer, a therapeutically effective amount of a M1 muscarinic receptor (M₁R) activator or a composition that upregulates gene expression of CHRM1 in the subject having cancer, and administering, to the subject having cancer, a therapeutically effective amount of a M3 muscarinic receptor (M₃R) inhibitor or a composition that downregulates gene expression of CHRM3 in the subject having cancer.
Embodiment 2: The method of embodiment 1, wherein the M₁R activator is a selective M₁R activator.
Embodiment 3: The method of embodiment 2, wherein the selective M₁R activator is Xanomeline or McN-A-343.
Embodiment 4: The method of embodiments 1-3, wherein the M₃R inhibitor is a selective M₃R inhibitor
Embodiment 5: The method of embodiment 4, wherein the selective M₃R inhibitor is Darifenacin
Embodiment 6: The method of embodiments 1-5, wherein the subject has a high CHRM1:3 ratio.
Embodiment 7: The method of embodiments 1-6, wherein the cancer is colorectal cancer, pancreatic cancer, or gastric cancer.
Embodiment 8: The method of embodiments 1-7, wherein treating cancer results in a decrease in proliferation of cancer cells in the subject.
Embodiment 9: The method of embodiments 1-8, wherein a composition that downregulates gene expression of CHRM3 is a composition that knocks out all or a portion of CHRM3.
Embodiment 10: The method of embodiment 9, wherein a composition that knocks out all or a portion of CHRM3 comprises a CRISPR-Cas9 system that targets CHRM3.
Embodiment 11: The method of embodiments 1-8, wherein a composition that downregulates gene expression of CHRM3 is a small interfering RNA or short hairpin RNA that targets CHRM3 mRNA.
Embodiment 12: The method of embodiments 1-8, wherein a composition that upregulates gene expression CHRM1 is a transcription activator that activates a CHRM1 promoter.
Embodiment 13: A method of reducing cancer cell proliferation comprising contacting one or more cancer cells with a combination therapy, wherein the combination therapy comprises: a M1 muscarinic receptor (M₁R) activator or a composition that upregulates gene expression of CHRM1; and a M3 muscarinic receptor (M₃R) inhibitor or a composition that downregulates gene expression of CHRM3.
Embodiment 14: The method of embodiment 13, wherein the cancer cells are in a subject.
Embodiment 15: The method of embodiment 14, wherein contacting one or more cancer cells comprises administering, to the subject, a therapeutically effective amount of a M1 muscarinic receptor (M₁R) activator or a composition that upregulates gene expression of CHRM1 in the subject, and administering, to the subject, a therapeutically effective amount of a M3 muscarinic receptor (M₃R) inhibitor or a composition that downregulates gene expression of CHRM3 in the subject.
Embodiment 16: The method of embodiments 13-15, wherein the M₁R activator is a selective M₁R activator.
Embodiment 17: The method of embodiment 16, wherein the selective M₁R activator is Xanomeline or McN-A-343.
Embodiment 18: The method of embodiments 13-17, wherein the M₃R inhibitor is a selective M₃R inhibitor.
Embodiment 19: The method of embodiment 18, wherein the selective M₃R inhibitor is Darifenacin.
Embodiment 20: The method of embodiments 14-19, wherein the subject has a high CHRM1:3 ratio.
Embodiment 21: The method of embodiments 13-20, wherein the cancer cells are colorectal cancer cells, pancreatic cancer cells, and gastric cancer cells.
Embodiment 22: The method of embodiments 13-21, wherein a composition that downregulates gene expression of CHRM3 is a composition that knocks out all or a portion of CHRM3.
Embodiment 23: The method of embodiment 22, wherein a composition that knocks out all or a portion of CHRM3 comprises a CRISPR-Cas9 system that targets CHRM3.
Embodiment 24: The method of embodiments 13-21, wherein a composition that downregulates gene expression of CHRM3 is a small interfering RNA or short hairpin RNA that targets CHRM3 mRNA.
Embodiment 25: The method of embodiments 13-21, wherein a composition that upregulates gene expression CHRM1 is a transcription activator that activates a CHRM1 promoter.
Embodiment 26: A method of increasing efficacy of a combination therapy in a subject having cancer comprising identifying a subject having cancer as having a high CHRM1:3 ratio; and administering the combination therapy to the subject having cancer, wherein the combination therapy comprises a therapeutically effective amount of a M1 muscarinic receptor (M₁R) activator and a therapeutically effective amount of a M3 muscarinic receptor (M₃R) inhibitor, wherein administering the combination therapy to subjects having cancer identified as having a high CHRM1:3 ratio increases efficacy of the combination therapy compared to administering the combination therapy to subjects having cancer identified as having a low CHRM1:3 ratio.
Embodiment 27: The method of embodiment 26, wherein the M₁R activator is a selective M₁R activator.
Embodiment 28: The method of embodiment 27, wherein the selective M₁R activator is Xanomeline or McN-A-343.
Embodiment 29: The method of embodiments 26-28, wherein the M₃R inhibitor is a selective M₃R inhibitor.
Embodiment 30: The method of embodiment 29, wherein the selective M₃R inhibitor is Darifenacin.
Embodiment 31: The method of embodiments 26-30, wherein the cancer is colorectal cancer, pancreatic cancer, or gastric cancer.
Embodiment 32: The method of embodiments 26-31, wherein an increased efficacy of the combination therapy results in a decrease in proliferation of cancer cells in the subject.
Embodiment 33: A method of potentiating effects of M1 muscarinic receptor (M₁R) activation in a subject having cancer comprising administering, to the subject, a therapeutically effective amount of a M1 muscarinic receptor (M₁R) activator or a composition that upregulates gene expression of CHRM1 in the subject, and administering, to the subject, a therapeutically effective amount of a M3 muscarinic receptor (M₃R) inhibitor or a composition that downregulates gene expression of CHRM3 in the subject, wherein the combination of activating M₁R and inhibiting M₃R potentiates M₁R activation.
Embodiment 34: The method of embodiment 33, wherein the M₁R activator is a selective M₁R activator.
Embodiment 35: The method of embodiment 34, wherein the selective M₁R activator is Xanomeline or McN-A-343.
Embodiment 36: The method of embodiments 33-35, wherein the M₃R inhibitor is a selective M₃R inhibitor.
Embodiment 37: The method of embodiment 36, wherein the selective M₃R inhibitor is Darifenacin.
Embodiment 38: The method of embodiments 33-37, wherein the subject has a high CHRM1:3 ratio.
Embodiment 39: The method of embodiments 33-38, wherein the cancer is colorectal cancer, pancreatic cancer, or gastric cancer.
Embodiment 40: The method of embodiments 33-39, wherein potentiating effects of M₁R activation results in a greater decrease in proliferation of cancer cells in the subject having cancer compared to a subject having cancer administered only a therapeutically effective amount of a M₁R activator.
Embodiment 41: The method of embodiments 33-40, wherein a composition that downregulates gene expression of CHRM3 is a composition that knocks out all or a portion of CHRM3.
Embodiment 42: The method of embodiment 41, wherein a composition that knocks out all or a portion of CHRM3 comprises a CRISPR-Cas9 system that targets CHRM3.
Embodiment 43: The method of embodiments 33-40, wherein a composition that downregulates gene expression of CHRM3 is a small interfering RNA or short hairpin RNA that targets CHRM3 mRNA.
Embodiment 44: The method of embodiments 33-40, wherein a composition that upregulates gene expression CHRM1 is a transcription activator that activates a CHRM1 promoter.

### EXAMPLES

### A. Example 1

Colorectal cancer (CRC) is the major cause of cancer death in men under age 50, accounting for ˜10% of cancers in Veterans—yearly the VA cares for >175,000 Veterans with CRC. Current therapies for advanced CRC provide transient, if any, prolongation of life—novel approaches are urgently needed. This study addresses this urgent need and will fill knowledge gaps regarding the mechanisms underlying the role muscarinic receptors play in CRC. Seminal findings from previous research revealed that acetylcholine, produced by neurons and CRC cells, activates M3 muscarinic receptors (M₃R)—thereby stimulating CRC progression. In cell and animal models, deleting or blocking activation of M₃R attenuates CRC. A surprising finding showed that, in contrast to the pro-neoplastic effects of activating M₃R, activating M₁R robustly attenuates CRC cell proliferation. Even more exciting is the finding that activating M₁R while blocking M₃R activation potentiates this effect, a finding with important therapeutic implications. The balance between the genes encoding M₁R and M₃R, the CHRM1:3 ratio, predicted treatment responses. Jointly targeting M₁R and M₃R may arrest colon cancer progression more effectively than targeting either receptor alone, and the proposed work opens the door to novel biomarkers and potentially transformative CRC therapy.

Disclosed herein are methods that derive from the concepts that targeting both M₁R and M₃R activity potentiates CRC therapeutic responses and the balance of M₁R to M₃R expression modulates both CRC progression and responses to this treatment approach. In some aspects, the disclosed methods derive from: (a) measuring CHRM1:3 ratios in established and primary patient-derived cancer cell lines; (b) using state-of-the-art xenograft and mouse models that mimic human disease; (c) unique conditional knockout mice with intestinal epithelial cell-selective M₁R and M₃R deficiency; (d) access to M1-DREADD mice wherein M₁R can be selectively activated only by clozapine N-oxide; and (e) the use of single-cell RNA sequencing to gain novel mechanistic insights. Clinical impact derives from the great potential of treating advanced CRC jointly with repurposed, FDA-approved M₁R activators and M₃R inhibitors, alone or in combination with existing therapy, and identifying the CHRM1:3 ratio as a biomarker of treatment responses.

Colorectal cancer comprises 10% of all cancers in U.S. Veterans. Moreover, 40% of Veterans have Stage III-IV disease for which there are few effective treatments. Sophisticated cell and animal models will be utilized to determine if the balance between the expression by human colon cancer cells of M1 and M3 muscarinic receptors provides a novel biomarker that predicts the likelihood of responding to repurposed FDA-approved drugs to activate M1 receptors while blocking M3 receptor activation.

Colorectal cancer (CRC) is frequently diagnosed at advanced stages when limited therapeutic options provide only transient benefit. In 2024, >50,000 Americans, many of them Veterans, died from CRC, the deadliest cancer in young men. As the tumor microenvironment plays an important role in colon neoplasia, previous work focuses on finding therapeutic targets in the enteric nervous system. Previously, cholinergic muscarinic neurotransmission was identified as such a target. Activated M3 muscarinic receptors (M₃R) induce the expression of selective matrix metalloproteinases (MMP1, 7, 10) and microRNAs (miR21, 221, 222) that promote CRC progression. In human CRC cells, downregulating expression of CHRM3, the gene encoding M₃R, or blocking M₃R activation, robustly attenuates cell proliferation, survival, and migration. In murine CRC models, deleting M₃R expression greatly diminishes colon tumor formation.

In contrast to overexpression of M₃R, M₁R expression is surprisingly diminished in CRC. Consistent with this finding, selectively activating M₁R robustly inhibits CRC cell proliferation, an action diametrically opposed to what happens when M₃R is activated. Jointly targeting M₁R and M₃R may arrest CRC progression more effectively than targeting either receptor alone, which opens the door for potentially transformative therapy. Exciting preliminary data support this idea. It was previously showed that CRC cell lines differ in expression levels of the genes encoding M₁R (CHRM1) and M₃R (CHRM3), a trait designated as the CHRM1:3 ratio. The data herein reveal that whereas treating CRC cells with a selective activator of MIR dose-dependently blocks cell proliferation, doing so with a combination of FDA-approved drugs to selectively activate M₁R and inhibit M₃R potentiates this effect. This drug combination is most potent in cells with a high CHRM1:3 ratio. Leveraging these reciprocal relationships offers the potential to reduce doses of drugs needed to activate M₁R while inhibiting M₃R activation, thus mitigating off-target cholinergic side-effects.

This therapeutic approach can be explored using in vitro, ex vivo, and in vivo methods and unique mouse models. The short-term goals are to define the benefits of joint M₁R activation/M₃R inhibition in preclinical models and the value of the CHRM1:3 ratio as a biomarker predicting response. Genetic and transcriptomic studies can shed light on the molecular basis for the opposing actions of M₁R and M₃R.

### 1. Approach:

i. Define the Efficacy of Joint M₁R activation/M₃R Inhibition on Key Attributes of Human CRC Cells and the Predictive Value of the CHRM1:3 Expression Ratio

The CHRM1:3 ratio will be measured in 10 well-characterized CRC cell lines that will then serve as a test panel for further experiments. Using this CRC cell panel, the efficacy of selective M₁R activators and M₃R inhibitors will be compared, alone and combined, on CRC cell attributes—proliferation, survival, migration, and invasion. To test the potential of the CHRM1:3 ratio as a biomarker, its predictive value will be ascertained for each response. Combining M₁R activators with M₃R inhibitors may potentiate treatment responses, and the CHRM1:3 ratio, a potential clinical biomarker, may predict cellular susceptibility to this novel drug combination.

ii. Elucidate the Benefits of Targeting M₁R and M₃R and the Predictive Value of the CHRM1:3 Ratio Using CRC Xenografts

In vivo treatment responses can be determined using xenografts derived from CRC cell lines for which CHRM1:3 ratios were categorized previously. Additionally, the CHRM1:3 ratio can be measured and xenografts will be created using de-identified patient-derived primary CRC cell lines. To test therapeutic efficacy on xenograft growth and regression, mice harboring freshly injected CRC cells and previously established xenografts will be treated with selective M₁R activators and M₃R inhibitors, alone and combined. To identify divergent genetic and molecular pathways underlying the opposing actions of M₁R and M₃R activation; portions of excised xenografts can be used for RNA sequencing and confirm changes in gene and protein expression by qPCR and immunoblotting. The disclosed drug regimen can slow xenograft growth, promote xenograft regression, and validate the CHRM1:3 ratio as a biomarker predicting therapeutic responses.

iii. Test Joint Treatment with Selective M₁R activators and M₃R Inhibitors in Murine CRC Models.

Using established in vivo murine colon cancer models, the effects of treatment with M₁R activators and M₃R inhibitors can be quantified, alone and together, on CRC progression and regression. To uncover differential effects of treatments on molecular pathways, tumors can be analyzed using RNA-Seq and gene/protein analysis. To determine the mechanistic basis underlying this therapeutic approach and confirm unambiguously the roles of M₁R and M₃R; responses will be compared in wild-type, in M₁R and M₃R conditional knockout (CKO) mice, and in M1-DREADD mice that do not respond to conventional M₁R agonists (e.g., acetylcholine).

### B. Example 2

### 1. Summary:

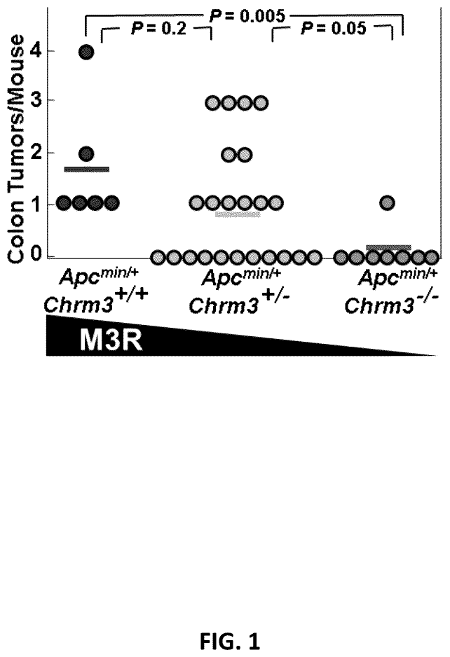

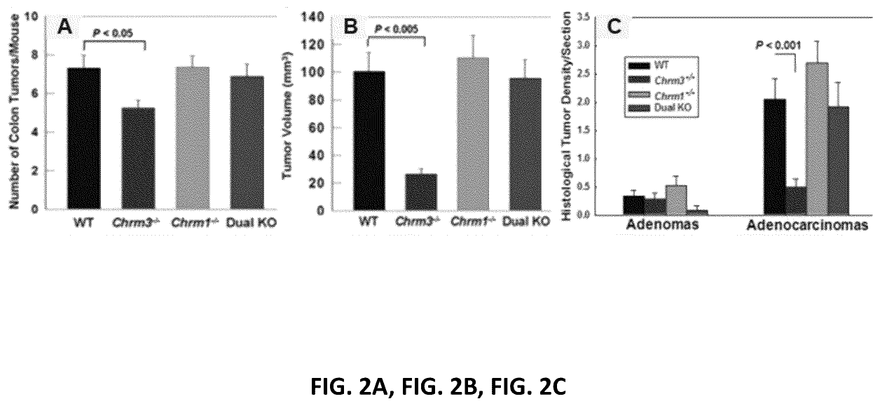

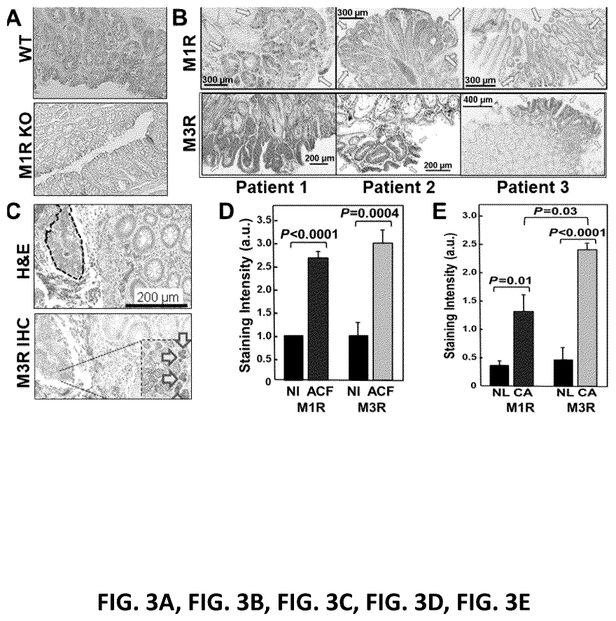

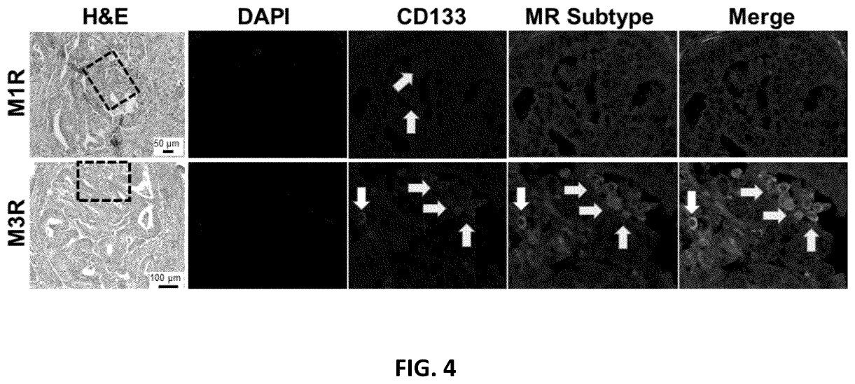

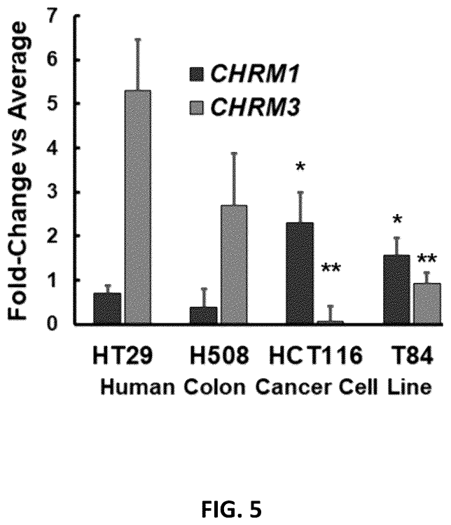

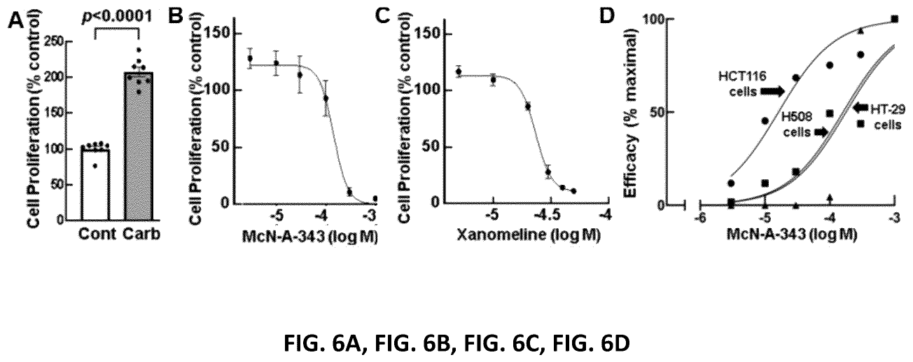

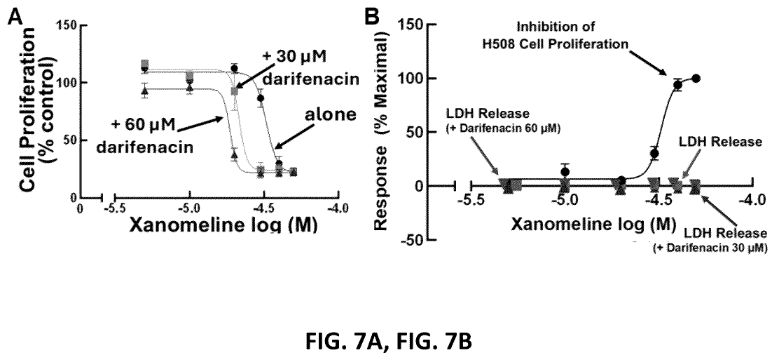

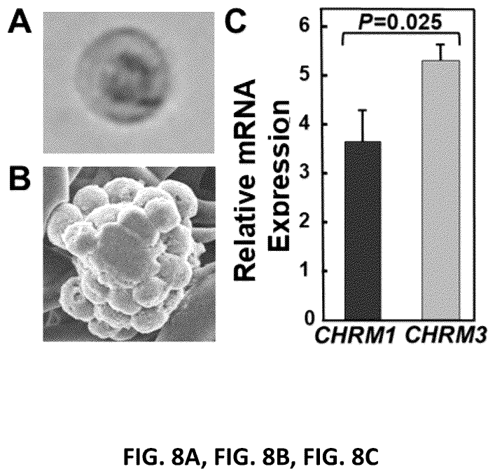

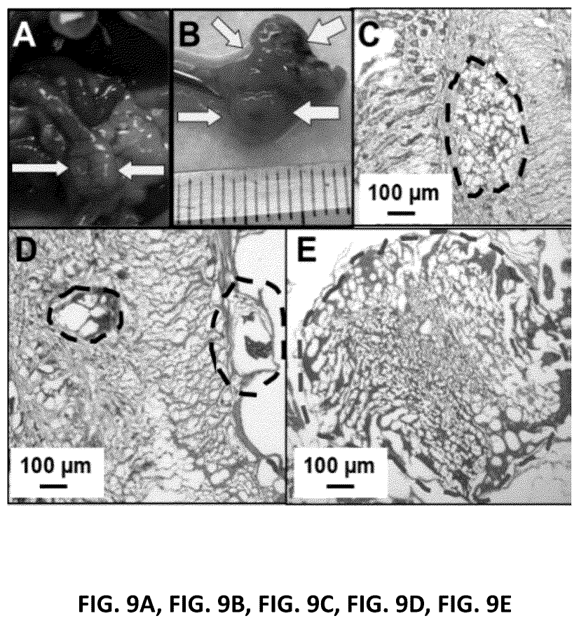

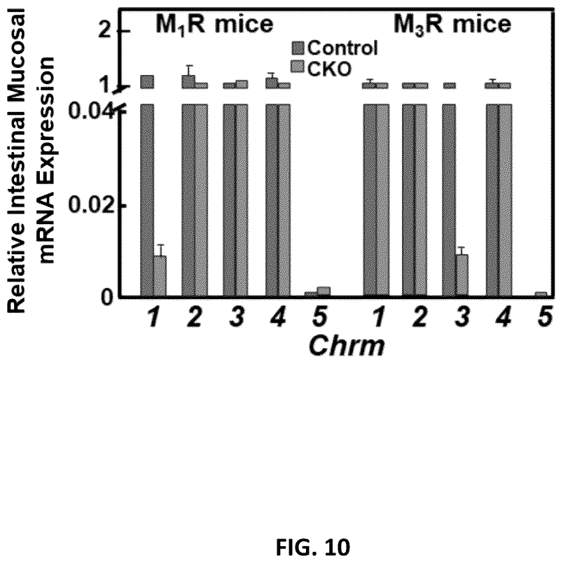

M₃R activators augment colon neoplasia and global M₃R deletion in mice attenuates colon tumorigenesis (FIGS. 1-2); strikingly, the latter finding is negated by concurrent global deletion of M₁R (FIG. 2). Additional key findings reveal that: (a) whereas Chrm3, the gene encoding M₃R, is overexpressed in human CRC, the opposite is true for Chrm1, the gene encoding M₁R; (b) M₃R expression levels progressively exceed those of M₁R as colon neoplasia advances from dysplasia to adenomas to invasive cancer (FIGS. 3-4); (c) M₃R but not M₁R is selectively overexpressed in CRC stem cells at the tumor invasive front, a region with activated β-catenin signaling (FIG. 4); (d) human CRC cells can be classified according to their relative expression of CHRM1 and CHRM3, a trait designated as the CHRM1:3 ratio (FIG. 5); and (e) tumoroids derived from human CRC stem cells maintain the CHRM1:3 ratio of parental cells (FIG. 8). These insights into CRC biology open the door to novel therapeutic interventions using selective M₁R activators and M₃R inhibitors (FIG. 6). Preliminary data show that combining a selective M₁R activator (xanomeline) with a selective M₃R inhibitor (darifenacin) more potently attenuates CRC cell proliferation than either agent acting alone (FIG. 7A) and does so without cell toxicity (FIG. 7B). In addition to murine CRC models, expansion of CRC stem cells as tumoroids while maintaining the parental CHRM1:3 ratio (FIG. 8), a novel orthotopic xenograft model (FIG. 9), the creation and breeding of conditional knockout (CKO) mouse colonies with intestinal epithelial cell-selective CHRM1/M₁R and CHRM3/M₃R deficiency (FIG. 10), and access to M1-DREADD mice provide unique assets. Collectively, these resources offer a singular opportunity to test a novel treatment strategy combining drugs that selectively activate M₁R and inhibit M₃R and at the same time explore the underlying mechanisms and the value of the CHRM1:3 ratio as a biomarker predicting responses.

### 2. Background

Within the tumor microenvironment (TME), neurotransmission via connections between the central (CNS) and enteric (ENS) nervous systems modulates colorectal cancer (CRC) progression. Seminal findings revealed that acetylcholine (ACh), a prominent CNS and ENS neurotransmitter, produced and released by CRC cells, activates muscarinic cholinergic receptors expressed by CRC cells. Of the five muscarinic receptor subtypes, designated M₁R-M₅R, it was demonstrated that activation of M₃R, encoded by CHRM3, drives CRC cell proliferation, survival, migration, and invasion. As anticipated from these findings, primary colon cancers overexpressing M₃R are more likely to metastasize. Notably, in murine CRC models, global M₃R deficiency robustly and dose-dependently attenuates intestinal neoplasia (FIG. 1; as depicted by the blue symbols, compared to six littermate controls only one of eight M₃R-deficient Apcmin/+ mice had colon tumors p<0.005). Key post-M₃R signaling that mediates cell invasion, a forerunner of metastasis, was identified. M₃R activation selectively induces expression of matrix metalloproteinase-1 (MMP1), a collagenase that degrades extracellular matrix and facilitates cell invasion. Blocking MMP1 expression and activation in vitro abolished ACh-induced CRC cell invasion into endothelial cell monolayers. M₃R activation was found to stimulate binding of βPak-interacting exchange factor (βPix) to 3-catenin. βPix, a Rho family Cdc42/Rac1 guanine nucleotide exchange factor, regulates key attributes of neoplastic cells—cytoskeletal dynamics, cell polarity, migration, adhesion. M₃R agonist-induced noncanonical binding of βPix to β-catenin augments β-catenin transcriptional activity and the expression of β-catenin target genes and proteins that promote colon neoplasia.

In contrast to M₃R, little was known regarding the role of the M1 muscarinic receptor subtype (M₁R) in neoplasia. M₁R, expressed by normal and neoplastic colonic epithelial cells, mediates normal intestinal mucosal growth. Because M₁R and M₃R share overlapping tissue distribution and signal transduction via Gαq, their physiological actions were considered to be the same. For example, it was necessary to delete both M₁R and M₃R to abolish muscarinic agonist-induced pepsinogen secretion from gastric chief cells. Thus, it was predicted that deleting both receptors would have similar or additive effects on colon neoplasia. Instead, it was surprising to find that deleting M₁R negated the benefits of M₃R deficiency; mice with whole-body M₁R and M₃R deletion had as many colon cancers as WT mice (FIG. 2). More surprisingly, M₁R-deficient mice had more cancers than WT mice (green bar, FIG. 2C).

The effects of treating CRC cells were tested with muscarinic receptor agonists that selectively activate only M₁R and created unique investigative resources like conditional knockout (CKO) mice with intestinal epithelial cell-selective deletion of M₁R and M₃R. As reviewed below, selective M₁R activation profoundly and dose-dependently attenuates CRC cell proliferation. The overarching goal is to leverage the opposing actions of M₁R and M₃R therapeutically. Using innovative techniques and models, the ability of selective M₁R activators and M₃R inhibitors will be tested, alone and combined, to not only retard, but reverse, CRC progression.

Within the heterogeneous population of cells in a colon tumor, CRC stem cells possess unique attributes allowing them to invade normal tissues and blood vessels and spread to other organs, the root cause of CRC mortality. Published and preliminary data provide evidence that the balance of CHRM1/M₁R and CHRM3/M₃R expression, a feature designated as the CHRM1:3 ratio, greatly impacts these attributes; M₃R activation stimulates whereas M₁R activation attenuates CRC cell proliferation. Applying state-of-the-art biochemical, molecular, and genetic approaches to innovative complementary in vitro, ex vivo, and in vivo models, will help determine unambiguously the therapeutic value of targeting both M₁R and M₃R in CRC.

Building on this exciting new data, the CHRM1:3 ratio will be measured in 10 established human CRC cell lines and primary CRC cells. Then, the effects of selective M₁R activators and M₃R inhibitors will be compared, alone and combined, on cell proliferation, survival, migration, and invasion and determine the predictive value of the CHRM1:3 ratio. Subsequently, the effects of selective M₁R activators and M₃R inhibitors will be tested, alone and combined, on established CRC cell- and primary tumor cell-derived allotopic and orthotopic xenografts. Besides exploring treatment effects on xenograft growth and regression and validating the CHRM1:3 ratio as a biomarker predicting treatment responses, the genetic and molecular pathways that underlie the divergent effects of M₁R and M₃R activation will be explored. Using unique in-vivo CRC animal models, the effects of systemic administration of selective M₁R activators and M₃R inhibitors will be compared, alone and combined, on colon tumor progression and regression. Testing this approach using WT, CHRM1, and CHRM3 CKO mice and M1-DREADD mice can unambiguously clarify the therapeutic potential of selectively targeting M₁R and M₃R. These clinically relevant studies can spur therapeutic trials of repurposed FDA-approved selective M₁R activators (e.g., xanomeline) and M₃R inhibitors (e.g., darifenacin) to treat CRC.

Disclosed herein are methods that derive from the concept that the balance of M₁R to M₃R activity has diametrically opposed actions in CRC, that targeting M₁R and M₃R activity potentiates CRC treatment responses, and that the CHRM1:3 ratio is a biomarker predicting CRC responses to this novel therapeutic approach. These conceptual advances have important practical implications. These implications indicate selectively targeting M₁R and M₃R activity in CRC, a novel concept, has great therapeutic potential. In some aspects, the disclosed methods derive from the unique validated assets that will be used to accomplish overarching goals, including: (a) measuring CHRM1:3 ratios in established, well-characterized CRC cell lines and primary patient-derived cancer cells; (b) state-of-the-art allotopic and orthotopic xenograft models; (c) unique CKO mice with intestinal epithelial cell-selective M₁R and M₃R deficiency; and (d) innovative M1-DREADD (Designer Receptor Exclusively Activated by Designer Drug) mice expressing M₁R that isn't activated by conventional agonists. In some aspects, the disclosed methods identify the CHRM1:3 ratio as a biomarker for the value of treating CRC with the combination of M₁R activators and M₃R inhibitors.

CRC is the leading GI cause of death and the major cause of cancer death in men younger than age 50. CRC accounts for ˜10% of all cancers in Veterans; each year the VA cares for >175,000 Veterans with CRC. Of 9,096 Veterans newly diagnosed with CRC at VAs from 2017 to 2021, ˜40% had Stage III-IV disease. Even if screening reduced mortality by 50-70%, CRC deaths would still equal or exceed those from pancreatic, renal, esophageal, and ovarian cancer. Current therapies for advanced to CRC provide transient, if any, prolongation of life; e.g., <5% of CRCs respond to immunotherapy. Previous findings indicate muscarinic receptor expression and activation are important for CRC progression and spread, and thus the CHRM1:3 ratio is a biomarker for metastatic potential and the efficacy of treatment with M₁R activators and M₃R inhibitors. The therapeutic potential of selectively targeting these receptors will be tested in preclinical animal models relevant to human disease using FDA-approved drugs and novel approaches. These findings will have important prognostic and therapeutic implications for Veterans and the general population with CRC.

The following data demonstrate the promise and feasibility of the in vitro, ex vivo, and in vivo approaches proposed. For in vitro studies, established human CRC cell lines and fresh cancers can be used. For ex vivo studies, CRC cells as 3-dimensional organoids (tumoroids) can be grown, thereby expanding the ability to measure and regulate gene expression and study disease-relevant cell functions. For in vivo studies, allo- and ortho-topic xenograft models can be used, including a model developed wherein human CRC cells are implanted in the mouse sigmoid colon and develop into solid, invasive tumors that metastasize to regional lymph nodes and the liver, a model that faithfully recapitulates human CRC progression.

M₁R and M₃R expression in progressive colon neoplasia. M₃R activation promotes CRC progression; CHRM3, a conditional oncogene, and its protein product, M₃R, are overexpressed in up to 80% of colon cancers. M₃R expression substantially augments neoplasia in mouse models of colon cancer (FIG. 1), but the role of M₁R has been less clear. By interrogating primary cancers and metastases, the frequency of CHRM3 mRNA expression in CRC underestimates M₃R protein expression, most likely because mRNA lability results in its degradation during tissue retrieval, storage, and processing. Whereas CHRM3 was overexpressed in 47% of CRCs, a >2-fold increased M₃R expression was observed in 80% of cancers. Notably, M₃R overexpression predicted the presence of lymph node and liver metastases.

As in CRC, M₃R is overexpressed ≥two-fold in 82% of tubular adenomas, indicating that M₃R is overexpressed early in the colon neoplasia continuum. To test this notion, specific anti-M₁R and anti-M₃R antibodies were used [Alomone AMR-010 and AMR-006 both validated by showing absent staining in M₁R- (FIG. 3A) and M₃R-deficient mouse tissue], and interrogated aberrant crypt foci (ACF), the earliest histologically detectable stage of colon neoplasia. M₃R staining was 3-fold greater in ACF compared to normal crypts (3.00±0.24 vs. 1.00±0.24 au, mean±SEM, P=0.0004; n=6 subjects with familial adenomatous polyposis (FIG. 3D) the image in the bottom right panel of FIG. 3B clearly shows increasing M₃R staining intensity with progressive dysplasia). M₁R staining intensity in ACF (2.62±0.09 vs. 1.00±0.16 au, P<0.0001; FIG. 3D) was less intense than for M₃R (FIG. 3B).

Next, M₁R and M₃R staining was compared in eight cancers vs matched adjacent normal colon. M₃R was chiefly over-expressed in cells at the tumor invasive front (FIG. 3C). M₁R vs M₃R staining intensity in cancer was 1.41±0.34 au, mean±SEM, vs 2.38±0.23 au (P=0.03; FIG. 3E). M₁R staining intensity was ˜20% less than that for M₃R in ACF but ˜50% less than that for M₃R in cancer. To investigate the difference in M₃R overexpression in cells at the cancer invasive front, IF/confocal microscopy was used to examine M₁R and M₃R expression in CD133-positive CRC stem cells in colon cancer vs adjacent normal colon. At the tumor invasive front, CD133+-stained cells clustered around blood vessels had intense M₃R but no M₁R staining (FIG. 4). These findings highlight the imbalance between M₁R and M₃R staining as neoplasia progresses from normal colon epithelium in which M₁R and M₃R expression is consistently similar, to ACF where M₃R expression starts to predominate, to adenomas and cancer core and invasive edge cells where the gap between high M₃R and low M₁R expression progressively widens. Supporting these findings, the unbiased analysis of 78 paired CRC samples showed detection consistent CHRM1 downregulation. This also provided mechanistic clues to be pursued in this study—CHRM1 expression correlated with expression levels for tumor suppressors (APC and SMAD4) and negatively with levels of a key tumor promoter, CTNNB1, which encodes 0-catenin, and those of several j3-catenin target genes.

i. Comparison of CHRM1 to CHRM3 Expression in CRC Cells—Concept of the CHRM1:3 Ratio

Pursuing the findings described above, CHRM1 and CHRM3 expression was measured and compared in four commonly used human CRC cell lines, HT-29, H508, HCT116, and T84 cells. HT-29 and H508 cells were utilized for many in-vitro studies; HCT116 and T84 cells were used by other investigators. These well-characterized CRC cell lines all have mutated APC and/or CTNNB148, but their relative expression of CHRM1 and CHRM3 was not previously examined. As shown in FIG. 5, substantially higher levels of CHRM3 were detected in HT-29 and H508 cells. In contrast, T84 and HCT116 cells, more robustly express CHRM1 (FIG. 5). This suggested that CRC cell lines can be characterized by the CHRM1:3 ratio. Because of modest fluctuation in these relative values, it more useful to characterize the CHRM1:3 ratio as high (e.g., HCT116 cells), intermediate (e.g., T84 cells), or low (e.g., HT-29 and H508 cells).

ii. Drugs Activating M1R Attenuate Human CRC Cell Proliferation—Predictive Value of the CHRM1:3 Ratio

H508 human CRC cells which are CHRM1:3-low, responded to carbachol, a non-selective muscarinic agonist, with a two-fold increase in cell proliferation (FIG. 6A). In contrast, treating cells with two validated orthosteric modulators of M₁R activation, MCN-A-343 and xanomeline, robustly and dose-dependently attenuated cell proliferation (FIGS. 6B-C). Notably, the dose-response for McN-A-434-induced inhibition of CRC cell proliferation was shifted substantially to the left in HCT116 compared to H508 and HT-29 cells; half-maximal effective concentrations (EC50) were 7.89 μM for HCT116 cells, 136.8 μM for H508 cells, and 548.1 μM for HT-29 cells (FIG. 6D). The two orders of magnitude increase in potency for the M₁R activator in HCT116 vs HT-29 cells were credited to the fact that HCT116 cells are CHRM1:3-high 13 (FIG. 5), a concept tested in the current study.

iii. Potentiated Response with M₁R activator combined with M₃R Inhibitor

To determine if activating M₁R while inhibiting M₃R activation more potently inhibits CRC cell proliferation than either agent acting alone, two concentrations of darifenacin, a selective M₃R inhibitor, with increasing doses of xanomeline, a selective M₁R activator were combined. As shown in FIG. 7A, incubating H508 CRC cells with 30 and 60 μM darifenacin shifted the xanomeline dose-response progressively leftwards; half-maximal inhibitory concentrations (IC50) for xanomeline alone and xanomeline plus 30 and 60 μM darifenacin were 32.8, 21.6, and 18.7 μM, respectively (p<0.001). These data support the concept that simultaneously activating M₁R while inhibiting M₃R activation more potently attenuates CRC cell proliferation than either agent acting alone. To exclude cell toxicity with these drug concentration, cellular release of lactate dehydrogenase (LDH) was measured. As shown in FIG. 7B, no agent used, alone or in combination, altered cellular LDH release. In conclusion, the effects of these drugs on cell proliferation are mediated by post-muscarinic receptor signal transduction, not by cell toxicity.

iv. CRC Tumoroids Retain the Pro-Genitor Cell CHRM1:3 Ratio

Stem cells derived from HT-29 human CRC cells as tumoroid cultures that express in vivo phenotypes were isolated, purified, and expanded (FIG. 8A-B). Tumoroids remained viable through multiple passages (FIG. 8B) and maintained their CHRM1:3 status, e.g., both HT-29 cells (FIG. 5) and tumoroids express the CHRM1:3-low phenotype (FIG. 8C).

v. Orthotopic Xenograft Model Mimics Human CRC Progression

An orthotopic CRC xenograft model was created by injecting human and murine CRC cells submucosally into the sigmoid colon of mice. Within weeks, mice developed solid tumors (FIG. 9A-C), that invaded blood vessels and lymphatics (FIG. 9D) and spread to regional lymph nodes (FIG. 9E) and the liver, mimicking human CRC progression.

### C. Example 3

### 1. Introduction

Colorectal cancer (CRC) is the second most common cause of cancer death in the U.S. Increased expression of the M3 muscarinic receptor, M₃R, and its gene, CHRM3 is reported in CRC; in contrast, CHRM1/M₁R expression is reduced. Moreover, M₃R deficiency and blockade attenuates CRC development and progression, an effect negated by M₁R deficiency. In vitro, M₁R agonists inhibit human colon cancer cell proliferation. Based on these observations, it was hypothesized if concurrently blocking M₃R activation while stimulating M₁R activity might have therapeutic potential. This hypothesis of this study is inhibition of human colon cancer cell proliferation is augmented by combining selective M₃R antagonists with M₁R agonists.

### 2. Methods

Human colon cancer cell lines (H508, HCT116) were grown in complete culture medium in a 37° C. incubator with 5% CO2 and sub-cultured weekly. H508 or HCT116 cells were seeded in 96-well plates, allowed to adhere for 24 h and changed to serum-free culture medium. Cells were treated for 5 days with Xanomeline, a selective M₁R agonist, and were pre-incubated for 30 min with Darifenacin, a selective M₃R antagonist or Trospium, a non-selective MR antagonist. Cell proliferation and cytotoxicity were measured using the WST-1 and LDH colorimetric assays, respectively. Absorbance was measured at 440 nm and 490/680 nm, respectively, using a computer-interfaced plate reader.

### 3. Results

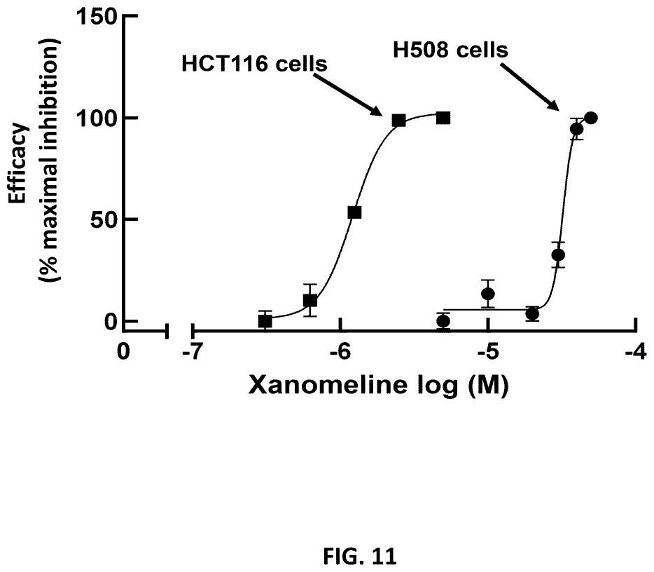

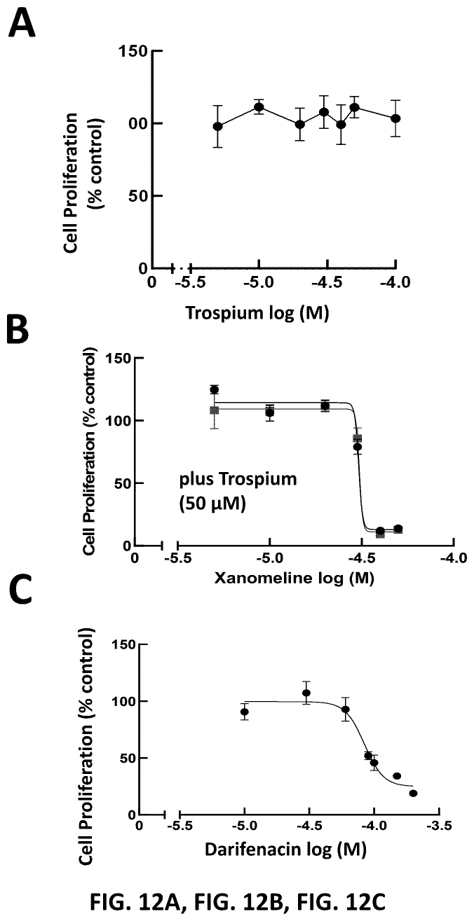

Relative CHRM1 and CHRM3 expression levels vary in commonly used human colon cancer cell lines. (FIG. 5). M₁R activation with xanomeline reduced human colon cancer cell proliferation in a dose-dependent manner. Xanomeline is a more potent inhibitor of proliferation in a cell line with higher levels of M₁R expression (FIG. 11). Selective M₃R, but not non-selective muscarinic receptor inhibition, augmented the anti-proliferative effects of M₁R agonism in human colon cancer cells (FIG. 12A-C, FIG. 7A). M₁R agonism with xanomeline and concurrent M₃R inhibition with darifenacin did not induce LDH leakage from human colon cancer cells (FIG. 7B).

### 4. Conclusions

These data support the concept that concomitant selective M₁R agonism and M₃R inhibition is a promising therapeutic strategy for colon cancer. The ratio of M₁R:M₃R expression in colon cancer may be a biomarker that predicts treatment response.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject having cancer comprising
administering, to the subject having cancer, a therapeutically effective amount of a M1 muscarinic receptor (M₁R) activator or a composition that upregulates gene expression of CHRM1 in the subject having cancer, and
administering, to the subject having cancer, a therapeutically effective amount of a M3 muscarinic receptor (M₃R) inhibitor or a composition that downregulates gene expression of CHRM3 in the subject having cancer.

2. The method of claim 1, wherein the M₁R activator is a selective M₁R activator.

3. The method of claim 2, wherein the selective M₁R activator is Xanomeline or McN-A-343.

4. The method of claim 1, wherein the M₃R inhibitor is a selective M₃R inhibitor.

5. The method of claim 4, wherein the selective M₃R inhibitor is Darifenacin.

6. The method of claim 1, wherein the subject has a high CHRM1:3 ratio.

7. The method of claim 1, wherein the cancer is colorectal cancer, pancreatic cancer, or gastric cancer.

8. The method of claim 1, wherein treating cancer results in a decrease in proliferation of cancer cells in the subject.

9. The method of claim 1, wherein a composition that downregulates gene expression of CHRM3 is a composition that knocks out all or a portion of CHRM3, a small interfering RNA or short hairpin RNA that targets CHRM3 mRNA.

10. The method of claim 1, wherein a composition that upregulates gene expression CHRM1 is a transcription activator that activates a CHRM1 promoter.

11. A method of reducing cancer cell proliferation comprising contacting one or more cancer cells with a combination therapy, wherein the combination therapy comprises:
a M1 muscarinic receptor (M₁R) activator or a composition that upregulates gene expression of CHRM1; and
a M3 muscarinic receptor (M₃R) inhibitor or a composition that downregulates gene expression of CHRM3.

12. The method of claim 11, wherein the cancer cells are in a subject.

13. The method of claim 12, wherein contacting one or more cancer cells comprises
administering, to the subject, a therapeutically effective amount of a M1 muscarinic receptor (M₁R) activator or a composition that upregulates gene expression of CHRM1 in the subject, and
administering, to the subject, a therapeutically effective amount of a M3 muscarinic receptor (M₃R) inhibitor or a composition that downregulates gene expression of CHRM3 in the subject.

14. The method of claim 11, wherein the M₁R activator is a selective M₁R activator.

15. The method of claim 14, wherein the selective M₁R activator is Xanomeline or McN-A-343.

16. The method of claim 11, wherein the M₃R inhibitor is a selective M₃R inhibitor.

17. The method of claim 16, wherein the selective M₃R inhibitor is Darifenacin.

18. The method of claim 12, wherein the subject has a high CHRM1:3 ratio.

19. The method of claim 11, wherein the cancer cells are colorectal cancer cells, pancreatic cancer cells, and gastric cancer cells.

20. A method of increasing efficacy of a combination therapy in a subject having cancer comprising
identifying a subject having cancer as having a high CHRM1:3 ratio; and
administering the combination therapy to the subject having cancer, wherein the combination therapy comprises a therapeutically effective amount of a M1 muscarinic receptor (M₁R) activator and a therapeutically effective amount of a M3 muscarinic receptor (M₃R) inhibitor,
wherein administering the combination therapy to subjects having cancer identified as having a high CHRM1:3 ratio increases efficacy of the combination therapy compared to administering the combination therapy to subjects having cancer identified as having a low CHRM1:3 ratio.
